(19) United States
(12) Patent Application Publication
(10) Pub. No.: US 2021/0055400 A1
(43) Pub. Date: Feb. 25, 2021

(54) Determining a Mixing Ratio in HVAC Systems

(71) Applicant: Siemens Schweiz AG, Zürich (CH)

(72) Inventors: Hilmar Konrad, Baar (CH); Karl-Heinz Petry, Reichenburg (CH); Tilman Weiers, Zug (CH)

(73) Assignee: Siemens Schweiz AG, Zürich (CH)

(21) Appl. No.: 17/000,284

(22) Filed: Aug. 22, 2020

(30) Foreign Application Priority Data
Aug. 22, 2019 (EP) 19193137.7
Apr. 9, 2020 (EP) 20169149.0

(51) Int. Cl. G01S 13/28; G01S 7/03; G01S 13/60

(52) U.S. Cl. CPC G01S 13/282; F25B 49/005; G01S 13/605; G01S 7/036

(57) ABSTRACT

Device for determining the mixing ratio of a mixture of at least two different fluids, the device comprising: a pipe section with a measuring region; wherein the mixture flows through the measuring region; a radar sensor system with a radar sensor chip arranged on an outer wall of the pipe section. The radar sensor system is configured to: irradiate frequency-modulated millimeter-radar waves (f_{S}) in a specified frequency range (Δf) into the measuring region; receive millimeter-radar waves (f_{R}) backscattered by the mixture; determine a frequency-dependent reflection coefficient (ρ_{f}) for the specified frequency range (Δf) using the backscattered millimeter-radar waves (f_{R}); and calculate or allocate the mixing ratio from the determined frequency-dependent reflection coefficient (ρ_{f}).

### CROSS-REFERENCE TO RELATED APPLICATIONS

This application claims priority to EP Application No. 20169149.0 filed Apr. 9, 2020 and EP Patent Application No. 19193137.7 filed Aug. 22, 2019, the contents of which are hereby incorporated by reference in their entirety.

### TECHNICAL FIELD

The present disclosure relates to HVAC systems. Various embodiments of the teachings herein include methods and/or systems for determining the mixing ratio of a fluid flowing through pipes in heating, ventilation, air conditioning and refrigeration systems by means of RADAR, measuring devices, and/or methods for determining the mixing ratio of a fluid.

### BACKGROUND

This disclosure relates, in particular, to determining mixing ratios in intelligent (smart) flow valves. A mixing ratio of a mixture of glycol and water often needs to be determined in this case. Knowledge of the glycol content in a mixture of water and glycol allows adequate processing of the heat transfer through the valve. international patent application WO 2012/065276 A1 relates to the determination of a heat flow of a heat-transporting fluid. According to WO 2012/065276 A1, two ultrasound transducers 14, 15 are arranged in a device 10 for measuring a heat flow. The ultrasound transducers communicate with a regulator 19. The regulator 19 is in turn connected to an evaluation unit 20. In addition, the device 10 comprises a temperature sensor 17, which is arranged between the two ultrasound transducers.

In the device 10 in WO 2012/065276 A1, the absolute temperature of a fluid is accordingly determined using the temperature sensor 10. At the same time, the speed of sound in the fluid is measured using the ultrasound transducers 14, 15. Density and mixing ratio of a water-glycol mixture can accordingly be inferred from the absolute temperature and the measured speed of sound.

Patent application DE 10 2007 015 609 A1 discloses a measuring device 2 with ultrasound measuring heads 4 for determining flow rates. The measuring device 2 also comprises two temperature probes 9 for detecting the temperature drop between the inlet flow end and the return flow end. The temperature probes 9 and the ultrasound measuring heads 4 are connected to a controller 12. The measuring device 2 in DE 10 2007 015 609 A1 provides a microanemometer 13. The microanemometer 13 is arranged between inlet flow side and return flow side and is likewise connected to the controller 12. An estimate k in respect of the specific heat results from the values detected by the microanemometer 13. The microanemometer 13 therefore allows values of k to be included in a heat flow estimate. It is conceivable to infer the composition of a water-glycol mixture from the values of k.

In addition to the approaches of DE 10 2007 015 609 A1 and WO 2012/065276 A1, a manual input is possible. Instead of automatically determining a mixing ratio, the manual approach requires an input by a user. The approach assumes sufficient knowledge of the mixing ratio of a water-glycol mixture in the pipes of a heating, ventilation and air conditioning system. The manual approach is susceptible to incorrect inputs by a user.

### SUMMARY

The present disclosure teaches classification of liquids, in particular of water-glycol mixtures, which manages without complex ultrasound sensors. A classification corresponding to the present disclosure avoids errors due to incorrect inputs by users. For example, some embodiments include a device for determining the mixing ratio of a mixture (FL), wherein the mixture (FL) comprises at least two different fluids (H2O, GLY), the device comprising: a pipe section (2) with a measuring region (MR), in particular one through which a fluid flows, provided for determining the mixing ratio; wherein the mixture (FL) is provided to flow through the pipe section (2); a radar sensor system (RS) comprising a radar sensor chip (RC), wherein the radar sensor chip (RC) has a sensor outer side, which is arranged on an outer wall of the pipe section (2) and/or penetrates this outer wall; wherein the radar sensor system (RS) is configured to: irradiate frequency-modulated millimeter-radar waves (f_{S}) in a specified frequency range (Δf) via the sensor outer side into the measuring region (MR); receive millimeter-radar waves (f_{R}) backscattered back at the mixture (FL); determine a frequency-dependent reflection coefficient (ρ_{f}) for the specified frequency range (Δf) using the backscattered millimeter-radar waves (f_{R}); and calculate the mixing ratio from the determined frequency-dependent reflection coefficient (ρ_{f}).

In some embodiments, the radar sensor chip (RC) is configured to: irradiate frequency-modulated millimeter-radar waves (f_{S}) in a specified frequency range (Δf) via the sensor outer side into the measuring region (MR); and receive millimeter-radar waves (f_{R}) backscattered at the mixture (FL).

In some embodiments, the radar sensor system (RS) comprises a microcontroller (MC); wherein the microcontroller (MC) is in operative communication with the radar sensor chip (RC); wherein the microcontroller (MC) is configured to: determine a frequency-dependent reflection coefficient (ρ_{f}) for the specified frequency range (Δf) using the backscattered millimeter-radar waves (f_{R}); and calculate the mixing ratio from the determined frequency-dependent reflection coefficient (ρ_{f}).

In some embodiments, the microcontroller (MC) is configured to: receive a detection result (DET) comprising measured values relating to the backscattered millimeter-radar waves (f_{R}); and determine a frequency-dependent reflection coefficient (ρ_{f}) for the specified frequency range (Δf) using the detection result (DET).

In some embodiments, the radar sensor system (RS) comprises a signal processor (SP); wherein the signal processor (SP) is in operative communication with the radar sensor chip (RC); wherein the signal processor (SP) is in operative communication with the microcontroller (MC); wherein the signal processor (SP) is configured to: receive from the radar sensor chip (RC) received data (RDAT) comprising digitized signals relating to the backscattered millimeter-radar waves (f_{R}); generate from the received data (RDAT) a detection result (DET), which comprises digitized signals of the received data (RDAT) processed to form measured values; send the detection result (DET) to the microcontroller (MC); wherein the microcontroller (MC) is configured to: receive the detection result (DET) from the signal processor (SP); and to determine a frequency-dependent reflection coefficient (ρ_{f}) for the specified frequency range (Δf) using the detection result (DET).

In some embodiments, the microcontroller (MC) is configured to: send control data (CSP) to the signal processor (SP); wherein the control data (CSP) comprises at least one instruction for the irradiation of frequency-modulated millimeter-radar waves (f_{S}) in the specified frequency range (Δf); wherein the signal processor (SP) is configured to: receive the control data (CSP) from the microcontroller (MC); generate at least one control signal (CRC) from the received control data (CSP), wherein the at least one control signal (CRC) comprises at least one variable selected from a frequency, a frequency deviation, a modulation method; send the at least one control signal (CRC) to the radar sensor chip (RC); wherein the radar sensor chip (RC) is configured to: receive the at least one control signal (CRC) from the signal processor (SP); as a result of receiving the at least one control signal (CRC), irradiate frequency-modulated millimeter-radar waves (f_{S}) in the specified frequency range (Δf) via the sensor outer side into the measuring region (MR); and wherein irradiation occurs as a function of the at least one variable comprised by the at least one control signal (CRC).

In some embodiments, the radar sensor chip (RC) has at its sensor outer side at least one transmitting antenna (Tx0, Tx1); wherein the radar sensor system (RS) is configured to: irradiate frequency-modulated millimeter-radar waves (RADAR) in a specified frequency range (Δf) via the sensor outer side into the measuring region (MR) using the at least one transmitting antenna (Tx0, Tx1).

In some embodiments, the radar sensor chip (RC) has at its sensor outer side at least one receiving antenna (Rx0-Rx3); wherein the radar sensor system (RS) is configured to: receive millimeter-radar waves (f_{R}) backscattered at the mixture (FL) using the at least one receiving antenna (Rx0-Rx3).

In some embodiments, the device further comprises: a radar wave-absorbing layer (4); and wherein the radar wave-absorbing layer (4) is arranged on an outer wall of the pipe section (2) and/or penetrates this outer wall.

In some embodiments, the radar wave-absorbing layer (4) comprises a layer of radar wave-absorbing material (RAM); and wherein the radar wave-absorbing material (RAM) is a radar wave-absorbing foam.

In some embodiments, the radar wave-absorbing layer (4) comprises a layer of radar wave-absorbing material (RAM); and wherein the layer of radar wave-absorbing material (RAM) comprises small balls, which are coated with carbonyl iron.

In some embodiments, the radar wave-absorbing layer (4) comprises a layer of radar wave-absorbing material (RAM); wherein the layer of radar wave-absorbing material (RAM) comprises polyurethane; and wherein the layer of radar wave-absorbing material (RAM) is preferably mixed with small balls of carbonyl iron and/or graphite.

In some embodiments, the radar sensor system (RS) is configured to: irradiate frequency-modulated millimeter-radar waves (fS) with wavelengths between three and seventeen millimeters in a specified frequency range (Df) via the sensor outer side into the measuring region (MR).

As another example, some embodiments include a method for determining the mixing ratio of a mixture (FL), wherein the mixture (FL) comprises at least two different fluids (H2O, GLY) and is provided for a technical process in a device or system, wherein the method comprises the following steps: irradiating continuously frequency-modulated millimeter-radar waves (f_{S}) millimeter-radar waves (f_{S}) with at least two different frequencies in a measuring region (MR) with the mixture (FL) during a measuring process; receiving continuously frequency-modulated millimeter-radar waves (fR) backscattered at the mixture (FL) during the measuring process; determining a frequency-dependent reflection coefficient (ρ_{f}) using the continuously frequency-modulated millimeter-radar waves (f_{R}) backscattered at the mixture (FL), and using the at least two different frequencies; and calculating the mixing ratio from the determined reflection coefficient (ρ_{f}).

In some embodiments, the method further comprises: continuous wave irradiating of a transmitting antenna signal (Tx0′) with millimeter-radar waves (f_{S}) into the measuring region (MR) with the mixture (FL) during the measuring process; wherein the irradiated millimeter-radar waves (f_{S}) have a specified frequency deviation; receiving correspondingly frequency-modulated millimeter-radar waves (f_{R}) backscattered at the mixture (FL) using a receiving antenna signal (Rx0′) during the measuring process; mixing the transmitting antenna signal (Tx0′) with the receiving antenna signal (Rx0′) to form an intermediate frequency signal; transforming the intermediate frequency signal into an associated frequency spectrum (SP); and determining the mixing ratio from the frequency spectrum (SP).

### BRIEF DESCRIPTION OF THE DRAWINGS

Various details will become accessible to the person skilled in the art with reference to the following detailed description. The individual embodiments do not limit the scope of the teachings herein. The drawings, which are attached to the description, may be described as follows:

FIG. 1 illustrates a pipe section with a radar sensor system incorporating teachings of the present disclosure;

FIG. 2 shows, like FIG. 1, a pipe section with a radar sensor system, wherein a layer of radar wave-absorbing material is attached opposite the radar sensor system incorporating teachings of the present disclosure;

FIG. 3 schematically illustrates the control and/or regulating units for the radar sensor system incorporating teachings of the present disclosure;

FIG. 4 shows further details of the radar sensor chip incorporating teachings of the present disclosure; and

FIG. 5 illustrates a correlation between reflection coefficient and frequency on the basis of a graph incorporating teachings of the present disclosure.

### DETAILED DESCRIPTION

A miniature radar sensor system is described in project Soli (https://atap.google.com/soli/, released on Aug. 6, 2019). That miniature radar sensor system was originally developed for gesture recognition. In some embodiments, instead of radar-supported movement detection of fingers for gesture recognition, a mixing ratio is determined. The sensor has side dimensions of ten millimeters versus eight millimeters (10 mm×8 mm). Millimeter-radar waves at sixty gigahertz (60 GHz) are used. The power consumption is three hundred milliwatts (300 mW). The range of the sensor is ten meters (10 m). Further technical details on the Soli sensor can be seen, inter alia, in an article by Jaime Lien, Nicholas Gillian, M. Emre Karagozler, Patrick Amihood, Carsten Schwesig, Erik Olson, Hakim Raja and Ivan Poupyrev. That article was published in July 2016 in ACM Transactions on Graphics, volume 35, number 4, article 142. The article bears the title Soli: Ubiquitous Gesture Sensing with Millimeter Wave Radar.

In some embodiments, there is a robust arrangement for classification of a water-glycol mixture. For this, a radar sensor system is arranged adjacent to a pipe. The radar sensor system is therefore physically separate from the fluid to be examined. In some embodiments, the system may be used to carry out the examination of a water-glycol mixture using commercially obtainable components. For this reason, a commercially obtainable radar sensor is drawn on. A classification according to the present disclosure is suitable for industrial use, for example in valves in heating, ventilation, and air conditioning technology.

In some embodiments, the system and/or method provides a determination of a mixing ratio, which can be applied to a wide variety of fluids. The disclosed classification is not limited to mixtures of water and glycol therefore. Instead, the classification is also suitable for identifying dangerous liquids and/or dangerous components in a mixture.

In some embodiments, there is a method and a device, wherein the method and the device use a digital arithmetic unit for exact calculation of a mixing ratio of a mixture of at least two fluids. It is an aim of the present disclosure, moreover, to provide a method and a device, wherein the method and the device largely use the arithmetic functions of a digital arithmetic unit for precise calculation of a mixing ratio of a mixture of at least two fluids.

In some embodiments, the system and/or device may be used to determine mixing ratios as accurately as possible. For this, an arrangement is provided, which suppresses disturbances in a pipe section due to reflections.

In some embodiments, the system and/or device may be used to identify device outages, such as valves in heating, ventilation, and air conditioning technology. For example, measured values obtained using the radar sensor can be checked for plausibility. Optionally, a signal is transmitted to a user, according to which a device is to be maintained or repaired. It is likewise possible, in the case of implausible measured values, to close a valve. This locks a heating, ventilation and air conditioning system.

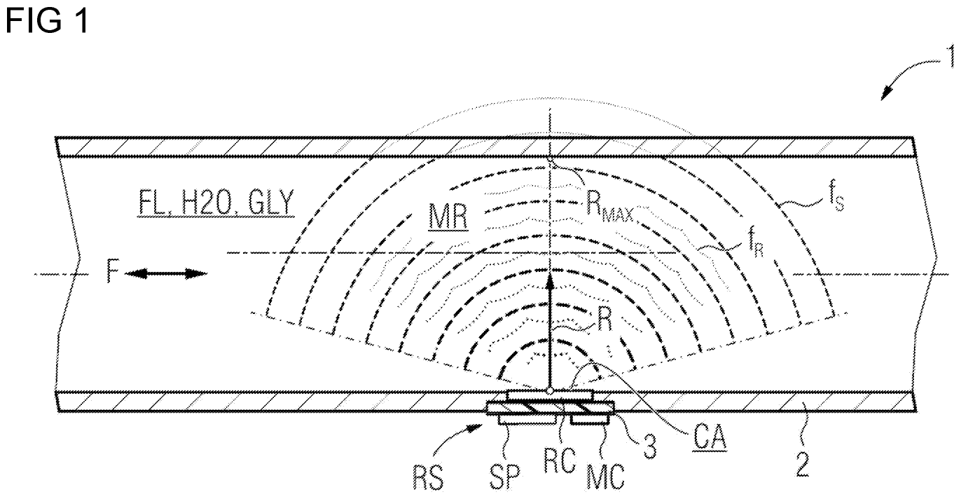

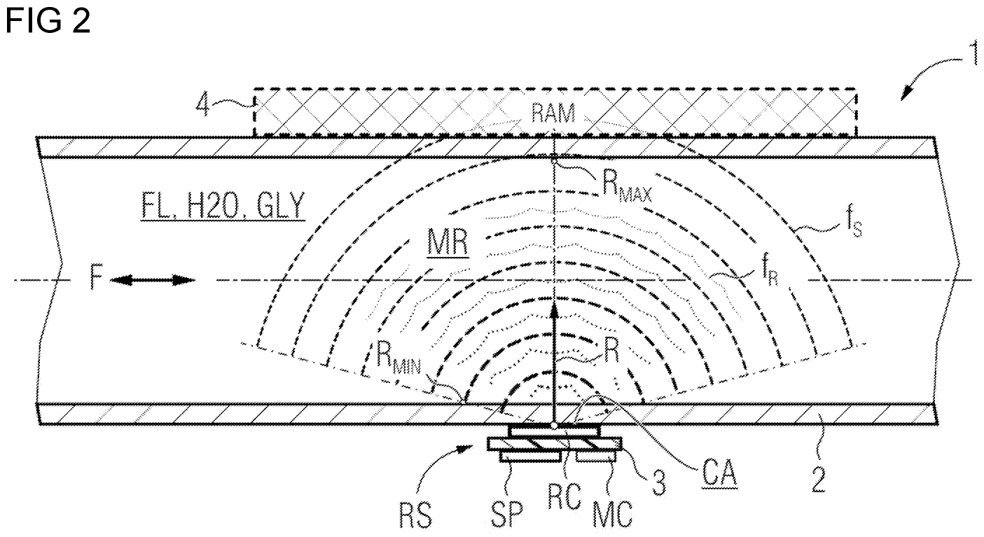

FIG. 1 illustrates the underlying measuring principle. Millimeter-radar waves f_{S} with a frequency of, for example, sixty Gigahertz and with a corresponding wavelength of five millimeters and less are irradiated into the interior MR of a pipe section 2. This interior MR can also be referred to as a measuring region or measuring space. The reference character R designates a radial distance of a sensor outer side CA of the radar sensor chip RC, in particular of the center of the surface of the sensor outer side CA. In anticipation of the following FIG. 2, R_{MIN} designates a minimum radial distance from which the millimeter-radar waves f_{S} emitted by the radar sensor chip RC run only through the mixture FL to be examined. R_{MAX} correspondingly designates a maximum radial distance, up to which the emitted millimeter-radar waves f_{S} run only through the mixture FL to be examined.

A miniaturized radar sensor chip RC is used in this connection. The radar sensor chip RC is located adjacent to the pipe section 2. The pipe section 2 itself is preferably produced from a material, which is substantially transparent for the above-mentioned millimeter-radar waves. The material can be, for example, a plastic material or a ceramic. A mixture FL, such as a mixture of water and glycol, flows through the pipe section 2. In the process the mixture FL scatters the millimeter-radar waves f_{S} irradiated into the interior MR of the pipe 2 or pipe section. The radar sensor chip RC receives the scattered millimeter-radar waves f_{R} and processes them in terms of signaling.

The scattering properties depend on the electromagnetic properties of the fluid FL. Accordingly, the mixture FL can be classified on the basis of its scattering properties.

For example, water and/or a water mixture are provided as the mixture FL. In particular, mixtures of water and at least one further substance selected from:
calcium chloride,
ethanol,
ethylene glycol,
glycerin,
potassium acetate,
potassium formiate,
magnesium chloride,
methanol,
sodium chloride and/or
1,2-propane diol
   are provided.

Furthermore, the fluid can comprise a coolant selected from:
R-401A,
R-404A,
R-406A,
R-407A,
R-407C,
R-408A,
R-409A,
R-410A,
R-438A,
R-500, and/or
R-502.

The preceding lists are not final.

What is known as a complex reflection coefficient ρ_{f} is analyzed. In particular, the changes in the complex reflection coefficient ρ_{f} with the material composition are analyzed. It is provided that the scattering properties of a fluid FL in the relevant frequency range are analyzed. Furthermore, attenuations of radio frequency signals provide indications of types of liquid. For example, a fluid such as milk can be distinguished from mains water in this way.

In some embodiments, changes in the dielectric properties of solutions with different glucose values can be identified. In this way it is possible to distinguish between different concentrations. Therefore, millimeter waves are suitable for glucose identification in biological media in concentrations similar to the blood sugar concentrations of diabetic patients.

In some embodiments, frequency-modulated millimeter-radar waves f_{S} are irradiated with a specified frequency deviation, in other words, in a specified frequency range Δf, within the meaning of a chirp signal into the measuring region MR. Such (continuously) frequency-modulated millimeter-radar waves f_{S} can be for example what are known as FMCW millimeter-radar waves f_{S}. The correspondingly frequency-modulated millimeter-radar waves f_{R} backscattered at the mixture FL and at the material of the pipe section 2 are then (down) mixed using a receiving antenna signal Rx0′ with the transmitting antenna signal Tx0′ to form an intermediate frequency signal. The intermediate frequency signal is then transformed into an associated frequency spectrum, such as by means of a Fourier transform. The frequency-dependent reflection coefficient ρ_{f} can then be determined from the frequency spectrum of the down-mixed intermediate frequency signal.

In some embodiments, simplified electronic further processing in a much lower frequency band is possible as a result of the down-mixing of the receiving antenna signal Rx0′. To minimize possible metrologically disadvantageous effects of reflections of the emitted millimeter-radar waves f_{S} on the material of the pipe section 2, for example a beginning of the intermediate frequency signal can be «cut away». The cut away signal corresponds from a time perspective to the radar waves f_{R} reflected by the wall of the pipe section 2 directly at the radar sensor chip RC (see FIG. 2). In other words, the time portion of the intermediate frequency signal, which can be assigned to reflected radar waves f_{R} within the minimum distance R_{MIN} from the sensor chip outer side, can be ignored. Correspondingly, the end of the intermediate frequency signal can be «cut off», and this corresponds from a time perspective to the radar waves f_{R} reflected by the opposing wall of the pipe section 2 (see FIG. 2). The time portion of the intermediate frequency signal, which can be assigned to reflected radar waves f_{R} larger than the maximum distance R_{MAX} from the sensor chip outer side, can be ignored.

In some embodiments, the complete intermediate frequency signal can be converted into the associated frequency spectrum. The frequency ranges in the frequency spectrum can then be ignored, which are directly proportional to the minimum distance R_{MIN} and maximum distance R_{MAX}. In the example of FIG. 2, radar waves f_{R} reflected at the mixture FL are only considered for radial distance values R—measured by the sensor chip outer side CA—, which are larger than the minimum distance R_{MIN} and smaller than the maximum distance R_{MAX}.

In some embodiments, a radar wave-absorbing layer 4 can be disposed. FIG. 2 shows such a radar wave-absorbing layer 4. The layer 4 suppresses disturbances. It can be arranged in such a way that it externally encloses at least parts of the pipe 2. The radar wave-absorbing layer 4 can also be arranged inside the pipe. In some embodiments, the wall or the wall of the pipe comprises a radar wave-absorbing material.

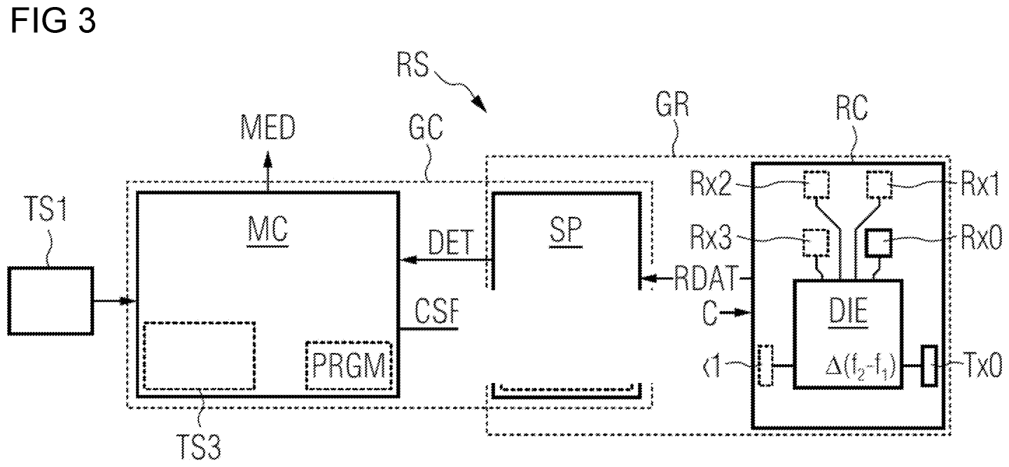

FIG. 3 shows a radar sensor system RS comprising a radar sensor chip with integrated signal processor GR. Radar sensor system RS also comprises a microcontroller with integrated signal processor GC. Using a first temperature sensor TS1, the microcontroller with integrated signal processor GC detects the temperature of a mixture FL in the pipe section 2. Using an interface, the microcontroller with integrated signal processor GC outputs digital or analog information relating to the type of mixture FL. In particular, the microcontroller with integrated signal processor GC outputs digital or analog information relating to the mixing ratio of the mixture FL.

For this purpose, a microcontroller MC comprised by the microcontroller with integrated signal processor GC sends control data CSP to a signal processor SP. In return the signal processor SP sends a detection result DET to the microcontroller MC. In some embodiments, the microcontroller with integrated signal processor GC also comprises the signal processor SP. In some embodiments, the microcontroller MC and the signal processor SP are arranged on the same chip. The microcontroller MC and the signal processor SP are in this case parts of a one-chip system.

In some embodiments, the microcontroller MC comprises a memory. For example, table values for determining the mixing ratio of a mixture FL, can be stored in the memory of the microcontroller MC. In some embodiments, the memory of the microcontroller MC is not volatile.

In some embodiments, the microcontroller MC has an arithmetic logic unit. The arithmetic logic unit of the microcontroller MC performs calculations, as are necessary, for example, for determining the mixing ratio of a mixture FL. The signal processor SP receives for its part data RDAT from the radar sensor chip RC. At the same time the signal processor SP controls the radar sensor chip RC using control signals CRC. It is therefore provided that the signal processor RC sends control signals CRC such as operating modes, frequencies and/or frequency deviation to the radar sensor chip RC.

In some embodiments, the radar sensor chip with integrated signal processor GR also comprises the signal processor SP. In some embodiments, the radar sensor chip RC and the signal processor SP are arranged on the same chip. The radar sensor chip RC and the signal processor SP are in this case parts of a one-chip system.

In some embodiments, the microcontroller MC and the signal processor SP and the radar sensor chip RC can be arranged on the same chip. The microcontroller MC and the signal processor SP and the radar sensor chip RC are in this case parts of a one-chip system.

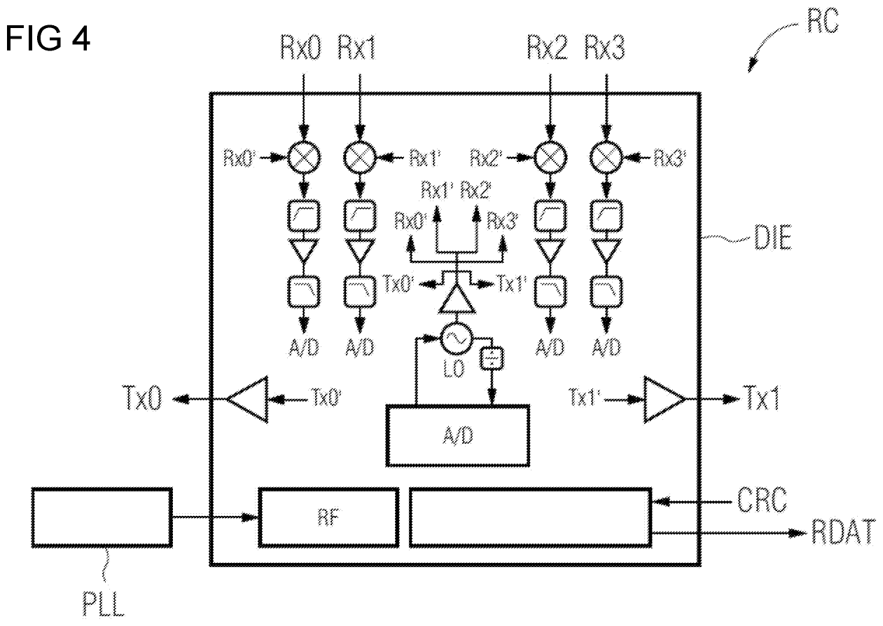

FIG. 4 illustrates details of the radar sensor chip RC. The radar sensor chip RC has at least one receiving antenna Rx0-Rx3. The at least one receiving antenna Rx0-RX3 is arranged to receive radiofrequency signals from the pipe section 2. The at least one receiving antenna Rx0-RX3 is in particular arranged for receiving millimeter-radar waves from the pipe section 2. In some embodiments, the radar sensor chip RC comprises at least two receiving antennas Rx0-RX3. Preferably, the radar sensor chip RC comprises even three or four receiving antennas Rx0-RX3.

The radar sensor chip RC also has at least one transmitting antenna Tx0, Tx1. The at least one transmitting antenna Tx0, Tx1 is arranged to irradiate radiofrequency signals into the pipe section 2. The at least one transmitting antenna Tx0, Tx1 is in particular arranged to irradiate millimeter-radar waves into the pipe section 2.

In some embodiments, the radar sensor chip RC comprises a radio frequency stage RF. The radio frequency stage RF communicates for its part with a phase locked loop PLL. That phase locked loop PLL can comprise a timer, moreover. In some embodiments, the radar sensor chip RC and the phase locked loop PLL are arranged on the same chip. The radar sensor chip RC and the phase locked loop PLL are in this case parts of a one-chip system.

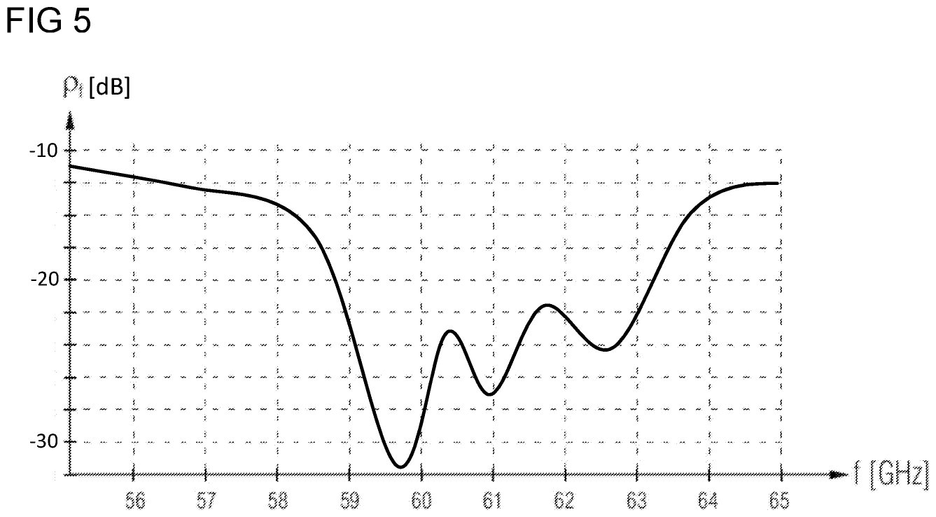

FIG. 5 shows an exemplary course of the reflection coefficient ρ_{f} over the frequency. The reflection coefficient ρ_{f} is used for determining the mixing ratio of the mixture FL. The reflection coefficient is defined as the ratio of reflected Vᵣ to irradiated signal Vₕ:

ρ_{f}=Vᵣ/Vₕ.

The reflected signal Vᵣ and the irradiated signal Vₕ are generally complex variables. For this reason, the value of the reflection coefficient |ρ_{f}| is frequently given as a function of the standing wave ratio SWR:

|ρ_{f}|=(SWR−1)/(SWR+1).

In some embodiments, the radar sensor system RS evaluates the value and/or the real part of the reflection coefficient ρ_{f}. For example, a mixing ratio can be assigned using the reflection coefficient ρ_{f} and using an assignment table stored in a memory of the radar sensor systems RS. An interpolation, in particular a linear interpolation, between table values is optionally used in addition to the stored table. In the sense used here the terms “approximately” and “substantially”, when they are used in connection with a numerical value or range, denote +/−5% of the stated numerical value or range.

The present disclosure therefore teaches a method for determining the mixing ratio of a mixture FL, wherein the mixture FL comprises at least two different fluids H2O, GLY and is provided in a device or system for a technical process, wherein the method comprises the following steps:
irradiating millimeter-radar waves f_{S} with at least two different frequencies in a measuring region MR with the mixture FL during a measuring process;
receiving millimeter-radar waves f_{R} backscattered at the mixture FL during the measuring process;
determining a frequency-dependent reflection coefficient ρ_{f} using the millimeter-radar waves f_{R} backscattered at the mixture FL, and using the at least two different frequencies; and
calculating the mixing ratio from the determined reflection coefficient ρ_{f}.

In some embodiments, the device or system comprises a heating, ventilation and/or air conditioning system. In some embodiments, the device or system also comprises a pipe section 2. The measuring region MR is ideally arranged in the pipe section 2.

In some embodiments, the method for determining the mixing ratio of a mixture FL comprises the following step:
allocating the determined reflection coefficient ρ_{f} to the mixing ratio of the mixture FL.

In some embodiments, the method for determining the mixing ratio of a mixture FL comprises the following step:
allocating the determined reflection coefficient ρ_{f} to the mixing ratio of the mixture FL using an assignment table.

The at least two different frequencies preferably differ by at least one megahertz, by at least two megahertz, and/or by at least five megahertz. Clearly different frequencies enable the determination of reflection coefficients ρ_{f} in an expanded frequency range Δf. Determination of the mixing ratio is more accurate therefore.

In some embodiments, the methods comprise the following steps:
irradiating continuously frequency-modulated millimeter-radar waves f_{S} in the measuring region MR with the mixture FL during the measuring process;
receiving continuously frequency-modulated millimeter-radar waves f_{R} backscattered at the mixture FL during the measuring process; and
determining a frequency-dependent reflection coefficient ρ_{f} using the continuously frequency-modulated millimeter-radar waves f_{R} backscattered at the mixture FL.

In some embodiments, there is a method for determining the mixing ratio of a mixture FL, wherein the mixture FL comprises at least two different fluids H2O, GLY and is provided in a device or system for a technical process, wherein the method comprises the following steps:
irradiating continuously frequency-modulated millimeter-radar waves (f_{S}) millimeter-radar waves (f_{S}) with at least two different frequencies in a measuring region (MR) with the mixture (FL) during a measuring process;
receiving continuously frequency-modulated millimeter-radar waves (fR) backscattered at the mixture (FL) during the measuring process;
determining a frequency-dependent reflection coefficient (ρ_{f}) using the continuously frequency-modulated millimeter-radar waves (f_{R}) backscattered at the mixture (FL), and using the at least two different frequencies; and
calculating the mixing ratio from the determined reflection coefficient (ρ_{f}).

In some embodiments, there is a method for determining the mixing ratio of a mixture FL, wherein the mixture FL comprises at least two different fluids H2O, GLY and is provided in a device or system for a technical process, wherein the method comprises the following steps:
irradiating continuously frequency-modulated millimeter-radar waves (f_{S}) millimeter-radar waves (f_{S}) in a measuring region (MR) with the mixture (FL) during a measuring process;
receiving continuously frequency-modulated millimeter-radar waves (fR) backscattered at the mixture (FL) during the measuring process;
determining a frequency-dependent reflection coefficient (ρ_{f}) using the continuously frequency-modulated millimeter-radar waves (f_{R}) backscattered at the mixture (FL), and
calculating the mixing ratio from the determined reflection coefficient (ρ_{f}).

In some embodiments, the methods comprise the following steps:
irradiating a chronological sequence of millimeter-radar waves f_{S} with radar frequencies f₁, f₂, fₙ that differ from each other into the measuring region MR with the mixture FL during the measuring process;
receiving millimeter-radar waves f_{R} backscattered at the mixture FL during the measuring process;
determining in each case one reflection coefficient ρ_{f} relating to at least two of the mutually different radar frequencies f₁, f₂, fₙ; and
calculating the mixing ratio from the respective reflection coefficients ρ_{f}.

In some embodiments, there is involvement of different radar frequencies f₁, f₂, fₙ, wherein the method comprises the following steps:
irradiating a chronological sequence of millimeter-radar waves f_{S} with at least five mutually different radar frequencies f₁, f₂, fₙ into the measuring region MR with the mixture FL during the measuring process; and
determining in each case one reflection coefficient ρ_{f} relating to at least five of the mutually different radar frequencies f₁, f₂, fₙ.

In some embodiments, the method for determining the mixing ratio of a mixture FL with the involvement of a sequence of millimeter-radar waves f_{S} comprises the following step:
irradiating a chronological sequence of millimeter-radar waves f_{S} each with mutually different radar frequencies f₁, f₂, fₙ into the measuring region MR with the mixture FL during the measuring process.

In some embodiments, the method for determining the mixing ratio of a mixture FL with the involvement of a sequence of millimeter-radar waves f_{S} comprises the following step:
irradiating a chronological sequence of millimeter-radar waves f_{S} with radar frequencies f₁, f₂, fₙ different from each other in pairs into the measuring region MR with the mixture FL during the measuring process.

In some embodiments, the methods comprise the following steps:
continuous wave irradiating of a transmitting antenna signal Tx0′ with millimeter-radar waves f_{S} into the measuring region MR with the mixture FL during the measuring process;
wherein the irradiated millimeter-radar waves f_{S} have a specified frequency deviation;
receiving correspondingly frequency-modulated millimeter-radar waves f_{R} backscattered at the mixture FL using a receiving antenna signal Rx0′ during the measuring process;
mixing the transmitting antenna signal Tx0′ with the receiving antenna signal Rx0′ to form an intermediate frequency signal;
transforming the intermediate frequency signal into an associated frequency spectrum SP; and
determining the mixing ratio from the frequency spectrum SP, at least from one frequency range within the frequency spectrum SP.

«Continuous wave irradiating» means that the transmitting antenna signal Tx0′ with the millimeter-radar waves f_{S} has a constant amplitude, at least a substantially constant amplitude. The irradiated millimeter-radar waves f_{S} with the specified frequency deviation is typically what are known as FMCW millimeter-radar waves (FMCW for frequency modulated continuous wave). A transmitting antenna signal Tx0′ of this kind is also called a chirp signal. In some embodiments, the frequency of the chirp signal continuously increases or decreases.

In some embodiments, a method for determining the mixing ratio of a mixture FL with the involvement of a signal mixing process comprises the following step:
transforming the intermediate frequency signal into an associated frequency spectrum SP.

In some embodiments, a method for determining the mixing ratio of a mixture FL with the involvement of a signal-mixing process comprises the following step:
Fourier transform of the intermediate frequency signal into an associated frequency spectrum SP.

In some embodiments, a method for determining the mixing ratio of a mixture FL with the involvement of a signal-mixing process comprises the following step:
transforming the intermediate frequency signal into an associated frequency spectrum SP using a fast Fourier transform.

In some embodiments, a method for determining the mixing ratio of a mixture FL with the involvement of a signal-mixing process comprises the following step:
assigning the frequency spectrum SP to a mixing ratio MV.

In some embodiments, a method for determining the mixing ratio of a mixture FL with the involvement of a signal-mixing process comprises the following step:
assigning the frequency spectrum SP to a mixing ratio using an assignment table.

In some embodiments, the millimeter-radar waves f_{S}, f_{R} are irradiated and received using a radar sensor systems RS attached to a pipe section 2. In some embodiments, the radar sensor system RS borders the measuring region MR. The pipe section 2 advantageously comprises the measuring region MR.

In some embodiments, the millimeter-radar waves f_{S}, f_{R} are irradiated and received using a radar sensor chip RC attached to a pipe section 2. In some embodiments, the radar sensor chip RC borders the measuring region MR. The pipe section 2 advantageously comprises the measuring region MR.

In some embodiments, there is a machine-readable medium with a set of instructions stored thereon, which on execution by one or more processor(s) cause the one or more processor(s) to carry out one of said methods. In some embodiments, the machine-readable medium is non-volatile.

In some embodiments, there is a computer program product with computer-executable instructions for carrying out one of the methods of this disclosure.

In some embodiments, there is a device for determining the mixing ratio of a mixture FL, wherein the mixture FL comprises at least two different fluids H2O, GLY, the device comprising:
a pipe section 2 with a measuring region MR, in particular one through which liquid flows, provided for determining the mixing ratio;
wherein the mixture FL is provided to flow through the pipe section 2;
a radar sensor system RS comprising a radar sensor chip RC, wherein the radar sensor chip RC has a sensor outer side, which is arranged on an outer wall of the pipe section 2 and/or penetrates this outer wall;
   wherein the radar sensor system RS is configured to:
irradiate frequency-modulated millimeter-radar waves (f_{S}) in a specified frequency range Δf via the sensor outer side into the measuring region MR;
receive millimeter-radar waves f_{R} backscattered at the mixture FL;
determine a frequency-dependent reflection coefficient ρ_{f} for the specified frequency range Δf using the backscattered millimeter-radar waves f_{R}; and
calculate the mixing ratio from the determined frequency-dependent reflection coefficient ρ_{f}.
In some embodiments, the radar sensor system RS can be configured to:
   irradiate frequency-modulated millimeter-radar waves f_{S} in a specified frequency range Δf via the sensor outer side into the measuring region MR during a measuring process;
   receive millimeter-radar waves f_{R} backscattered at the mixture FL during the measuring process.

In some embodiments, the sensor outer side penetrates the outer wall at least partially.

In some embodiments, the radar sensor system RS is configured to receive correspondingly frequency-modulated millimeter-radar waves f_{R} backscattered at the mixture FL.

In some embodiments, the pipe section 2 is part of a heating, ventilation and/or air conditioning system. In some embodiments, the pipe section 2 is part of a technical system or device. In some embodiments, the pipe section 2 comprises a valve. In some embodiments, the pipe section 2 can be a fluid path between inlet and outlet of the valve. In some embodiments, the pipe section 2 comprises an outer wall.

In some embodiments, the radar sensor system RS is configured to determine a frequency-dependent, dielectric reflection coefficient ρ_{f} for the specified frequency range Δf.

In some embodiments, the mixture FL comprises at least two different liquids H2O, GLY. The at least two different liquids H2O, GLY may be at a temperature of 293 kelvin and at a pressure of 1013 hectopascal in the liquid aggregate state.

In some embodiments, the radar sensor chip RC is configured to:
irradiate frequency-modulated millimeter-radar waves f_{S} in a specified frequency range Δf via the sensor outer side into the measuring region MR; and
receive millimeter-radar waves f_{R} backscattered at the mixture FL.

In some embodiments, there is a receiving radar sensor chip RC, wherein the receiving radar sensor chip RC is configured to:
receive correspondingly frequency-modulated millimeter-radar waves f_{R} backscattered at the mixture FL.

In some embodiments, the radar sensor system RS comprises a microcontroller MC;
wherein the microcontroller MC is in operative communication with the radar sensor chip RC;
   wherein the microcontroller MC is configured to:
determine a frequency-dependent reflection coefficient ρ_{f} for the specified frequency range Δf using the backscattered millimeter-radar waves f_{R}; and
calculate the mixing ratio from the determined frequency-dependent reflection coefficient ρ_{f}.

In some embodiments, the microcontroller MC is configured to:
calculate a real part of a frequency-dependent reflection coefficient ρ_{f} for the specified frequency range Δf using the backscattered millimeter-radar waves f_{R}; and
calculate the mixing ratio from the real part.

In some embodiments, the microcontroller MC is configured to:
calculate a value of a frequency-dependent reflection coefficient ρ_{f} for the specified frequency range Δf using the backscattered millimeter-radar waves f_{R}; and
calculate the mixing ratio from the value.

In some embodiments, the microcontroller MC is configured to:
calculate an imaginary part of a frequency-dependent reflection coefficient ρ_{f} for the specified frequency range Δf using the backscattered millimeter-radar waves f_{R}; and
calculate the mixing ratio from the imaginary part.

In some embodiments, there is a microcontroller MC, wherein the microcontroller MC is configured to:
receive a detection result DET comprising measured values relating to the backscattered millimeter-radar waves f_{R}; and
determine a frequency-dependent reflection coefficient ρ_{f} for the specified frequency range Δf using the detection result DET.

In some embodiments, the microcontroller MC is configured to receive from the radar sensor chip RC a detection result DET comprising digitized data relating to the backscattered millimeter-radar waves f_{R}.

In some embodiments, the microcontroller MC is configured to:
calculate a real part of a frequency-dependent reflection coefficient ρ_{f} for the specified frequency range Δf using the detection result DET; and
calculate the mixing ratio from the real part.

In some embodiments, the microcontroller MC is configured to:
calculate a value of a frequency-dependent reflection coefficient ρ_{f} for the specified frequency range Δf using the detection result DET; and
calculate the mixing ratio from the value.

In some embodiments, the microcontroller MC is configured to:
calculate an imaginary part of a frequency-dependent reflection coefficient ρ_{f} for the specified frequency range Δf using the detection result DET; and
calculate the mixing ratio from the imaginary part.

In some embodiments, the radar sensor system RS comprises a signal processor SP;
wherein the signal processor SP is in operative communication with the radar sensor chip RC;
wherein the signal processor SP is in operative communication with the microcontroller MC;
   wherein the signal processor SP is configured to:
receive from the radar sensor chip RC received data RDAT comprising digitized signals relating to the backscattered millimeter-radar waves f_{R};
generate from the received data RDAT a detection result DET, which comprises digitized signals of the received data RDAT processed to form measured values;
send the detection result DET to the microcontroller MC;
   wherein the microcontroller MC is configured to:
receive the detection result DET from the signal processor SP; and
determine a frequency-dependent reflection coefficient ρ_{f} for the specified frequency range Δf using the detection result DET.

In some embodiments, there is a microcontroller MC and signal processor SP:

wherein the microcontroller MC is configured to:
send control data CSP to the signal processor SP;
wherein the control data CSP comprises at least one instruction for the irradiation of frequency-modulated millimeter-radar waves f_{S} in the specified frequency range Δf;
   wherein the signal processor SP is configured to:
receive the control data CSP from the microcontroller MC;
generate at least one control signal CRC from the received control data CSP, wherein the at least one control signal CRC comprises at least one variable selected from
   a frequency,
   a frequency deviation,
   a modulation method;
send the at least one control signal CRC to the radar sensor chip RC;
   wherein the radar sensor chip RC is configured to:
receive the at least one control signal CRC from the signal processor SP;
as a result of receiving the at least one control signal CRC, irradiate frequency-modulated millimeter-radar waves f_{S} in the specified frequency range Δf via the sensor outer side into the measuring region MR; and
wherein irradiation takes place as a function of the at least one variable comprised by the at least one control signal CRC.

In some embodiments, the at least one control signal CRC describes at least one variable selected from
a frequency,
a frequency deviation,
a modulation method;
   and that
irradiation takes place as a function of the at least one variable described by the at least one control signal CRC.

In some embodiments, the signal processor SP is configured to:
generate the at least one variable as a function of the at least one instruction comprised by the control data CSP.

In some embodiments, the signal processor SP can be configured to:
calculate the at least one variable as a function of the at least one instruction comprised by the control data CSP.

In some embodiments, the modulation method describes at least one modulation method selected from
frequency modulation,
amplitude modulation,
phase modulation.

In some embodiments, the modulation method comprises at least one modulation method selected from
frequency modulation,
amplitude modulation,
phase modulation.

In some embodiments, the modulation method is at least a modulation method selected from
frequency modulation,
amplitude modulation,
phase modulation.

In some embodiments, the modulation method describes a frequency modulation or the modulation method comprises a frequency modulation or the modulation method is a frequency modulation.

In some embodiments, the radar sensor chip RC, on its sensor outer side, has at least one transmitting antenna Tx0, Tx1; wherein the radar sensor system RS is configured to:
irradiate frequency-modulated millimeter-radar waves f_{S} in a specified frequency range Δf via the sensor outer side into the measuring region MR using the at least one transmitting antenna Tx0, Tx1.

In some embodiments, the radar sensor chip RC is configured to:
irradiate frequency-modulated millimeter-radar waves f_{S} in a specified frequency range Δf via the sensor outer side into the measuring region MR using the at least one transmitting antenna Tx0, Tx1.

In some embodiments, the radar sensor chip RC has at its sensor outer side at least one receiving antenna Rx0-Rx3;

wherein the radar sensor system RS is configured to:
receive millimeter-radar waves f_{R} backscattered at the mixture FL using the at least one receiving antenna Rx0-Rx3.

In some embodiments, the radar sensor chip RC is configured to:
receive millimeter-radar waves f_{R} backscattered at the mixture FL using the at least one receiving antenna Rx0-Rx3.

In some embodiments, the at least one receiving antenna Rx0-Rx3 is different from the at least one transmitting antenna Tx0, Tx1. In a compact embodiment the at least one receiving antenna comprises the at least one transmitting antenna.

In some embodiments, the device additionally comprises:
a radar wave-absorbing layer 4; and
wherein the radar wave-absorbing layer 4 is arranged on an outer wall of the pipe section 2 and/or penetrates this outer wall.

In some embodiments, the radar wave-absorbing layer 4 penetrates the outer wall at least partially. The radar wave-absorbing layer 4 may be arranged on an outer wall of the pipe section 2 opposite the sensor outer side of the radar sensor chip RC. The radar wave-absorbing layer 4 serves to suppress disruptive reflections at the outer wall of the pipe section 2.

In some embodiments, the radar wave-absorbing layer 4 comprises a layer of radar wave-absorbing material (RAM). The radar wave-absorbing material (RAM) can be, in particular, a radar wave-absorbing foam. In some embodiments, the layer of radar wave-absorbing material (RAM) comprises small balls, which are coated, for example, with carbonyl iron. In some embodiments, the layer of radar wave-absorbing material (RAM) comprises polyurethane and is mixed with small balls of carbonyl iron and/or of (crystalline) graphite.

In some embodiments, there is a device for determining the mixing ratio of a mixture FL, wherein the mixture FL comprises at least two different fluids H2O, GLY, the device comprising:
a pipe section 2 with a first measuring region MR, in particular one through which liquid flows, provided for determining the mixing ratio and with a second measuring region MR, in particular one through which liquid flows, provided for determining the mixing ratio;
wherein the mixture FL is provided to flow through the pipe section 2 and a flow direction is defined thereby;
a first radar sensor system RS at the location of the first measuring region MR comprising a first radar sensor chip RC, wherein the first radar sensor chip RC has a first sensor outer side, which is arranged on an outer wall of the pipe section 2 and/or penetrates this outer wall;
a second radar sensor system RS at the location of the second measuring region MR comprising a second radar sensor chip RC, wherein the second radar sensor chip RC has a second sensor outer side, which is arranged on the outer wall of the pipe section 2 and/or penetrates this outer wall;
wherein the first and the second measuring region MR are arranged in series such that the first measuring region MR is located upstream of the second measuring region MR;
wherein a magnet is arranged at a region selected from the first and the second measuring region MR such that, due to the magnet, the magnetic flux penetrates the region and preferably penetrates perpendicular to the flow direction;
   wherein the first radar sensor system RS is configured to:
irradiate first millimeter-radar waves f_{S} in a first specified frequency range Δf via its first sensor outer side into the first measuring region MR;
receive first millimeter-radar waves f_{R} backscattered at the mixture FL;
determine a first reflection coefficient ρ_{f} using the backscattered first millimeter-radar waves f_{R};
   wherein the second radar sensor system RS is configured to:
irradiate second millimeter-radar waves f_{S} in a second specified frequency range Δf via its second sensor outer side into the second measuring region MR;
receive second millimeter-radar waves f_{R} backscattered at the mixture FL;
determine a second reflection coefficient ρ_{f} using the backscattered second millimeter-radar waves f_{R}; and
   wherein the device comprises an arithmetic unit in operative communication with the first and to the second radar sensor system RS, wherein the arithmetic unit is configured to:
calculate the mixing ratio by comparing the first reflection coefficient ρ_{f} with the second reflection coefficient ρ_{f}.

In some embodiments, the first sensor outer side penetrates the outer wall at least partially. In some embodiments, the second sensor outer side penetrates the outer wall at least partially.

In some embodiments, the magnet comprises a permanent magnet. In some embodiments, the magnet comprises an electromagnet. The magnet generates a maximum flux density in the pipe section 2 of at least 0.1 tesla, of at least 0.2 tesla or even 0.5 tesla. Higher flux densities allow more accurate determination of the mixing ratio.

In some embodiments, the first specified frequency range Δf comprises the second specified frequency range Δf. In particular, the first specified frequency range Δf can be equal to the second specified frequency range Δf. In some embodiments, the first specified frequency range Δf is different from the second specified frequency range Δf.

In some embodiments, the arithmetic unit is configured to:
receive the first reflection coefficient ρ_{f} from the first radar sensor system RS; and
receive the second reflection coefficient ρ_{f} from the second radar sensor system RS.

In some embodiments, the first radar sensor system RS is configured to send the first reflection coefficient ρ_{f} to the arithmetic unit. In some embodiments, the second radar sensor system RS is configured to send the second reflection coefficient ρ_{f} to the arithmetic unit.

In some embodiments, electromagnetic waves with wavelengths between two and thirty eight millimeters, electromagnetic waves with wavelengths between two and twenty five millimeters, and/or electromagnetic waves with wavelengths between three and seventeen millimeters, are considered as millimeter-radar waves f_{S}.

Parts of a device or a method according to the present disclosure can be implemented as hardware, as a software module, which is executed by an arithmetic unit, or using a Cloud computer, or using a combination of said possibilities. The software may comprise firmware, a hardware driver, which is implemented inside an operating system, or an application program. The present disclosure therefore also relates to a computer program product, which includes the features of this disclosure, or carries out the necessary steps. With an implementation as software, the described functions can be stored as one or more command(s) on a computer-readable medium. Some examples of computer-readable media include random access memory (RAM), magnetic random access memory (MRAM), read only memory (ROM), flash memory, electronically programmable ROM (EPROM), electronically programmable and erasable ROM (EEPROM), register of an arithmetic unit, a hard disk, a replaceable memory unit, an optical memory, or any suitable medium which can be accessed by a computer or by other IT devices and applications.

The above relates to various embodiments of the disclosure. Various changes to the embodiments can be made without deviating from the basic idea and without departing from the scope of this disclosure. The subject matter of the present disclosure is defined by its claims. A wide variety of changes can be made without departing from the scope of the following claims.

### LIST OF REFERENCE NUMERALS

1 measuring device
2 pipe section
3 circuit carrier, circuit board
4 radar wave-absorbing material (RAM)
CA chip outer side, transmitter side and receiver side
CRC control signals (for setting the operating modes, frequencies, frequency deviation)
CSP control data (for configuring the signal processor)
DET detection result (for the mixture)
DIE chip disks
F flow direction
FL fluid, mixture
f_{R} reflected millimeter-radar waves
f_{S} transmitted millimeter-radar waves
GC microcontroller with integrated signal processor
GLY glycol
GR radar sensor chip with integrated signal processor
H2O water
LO local oscillator
MC microcontroller
MED mixture type, medium type
MR measuring region, measuring space
PLL phase locked loop
PRGM computer program run on the microcontroller
PRGS computer program run on the signal processor
Pₜₑᵣₘ thermal output
R distance
RC radar sensor chip
RDAT received data (digitized data from the radar sensor chip)
RF radio frequency stage, RF front end
R_{MAX} maximum distance
R_{MIN} minimum distance
RS radar sensor system
Rx0-Rx3 receiving antennas
Rx0′-Rx3′ RF antenna signals, mixer signals
Tx0, Tx1 transmitting antennas
SP signal processor
SPI serial interface
SWR standing wave ratio
TS1 temperature sensor 1
TS2 temperature sensor 2
TS3 temperature sensor 3, integrated in the microcontroller
ρ_{f} frequency-dependent reflection coefficient

1. A device for determining the mixing ratio of a mixture comprising at least two different fluids, the device comprising:
a pipe section with a measuring region, wherein the mixture flows through the measuring region;
a radar sensor system including a radar sensor chip with a sensor outer side arranged on an outer wall of the pipe section;
wherein the radar sensor system is configured to:
irradiate frequency-modulated millimeter-radar waves in a specified frequency range into the measuring region;
receive millimeter-radar waves backscattered back from the mixture;
determine a frequency-dependent reflection coefficient for the specified frequency range using the backscattered millimeter-radar waves; and
calculate the mixing ratio from the determined frequency-dependent reflection coefficient.

2. The device as claimed in claim 1, wherein the radar sensor chip is configured to:
irradiate frequency-modulated millimeter-radar waves in a specified frequency range into the measuring region (MR); and
receive millimeter-radar waves backscattered at the mixture.

3. The device as claimed in claim 1, wherein:
the radar sensor system comprises a microcontroller in operative communication with the radar sensor chip;
the microcontroller is configured to:
determine a frequency-dependent reflection coefficient for the specified frequency range using the backscattered millimeter-radar waves; and
calculate the mixing ratio from the determined frequency-dependent reflection coefficient.

4. The device as claimed in claim 3, wherein the microcontroller is further configured to:
receive a detection result comprising measured values relating to the backscattered millimeter-radar waves; and
determine a frequency-dependent reflection coefficient for the specified frequency range using the detection result.

5. The device as claimed in claim 3, wherein:
the radar sensor system comprises a signal processor in operative communication with the radar sensor chip;
the signal processor is in operative communication with the microcontroller;
the signal processor is configured to:
receive from the radar sensor chip received data comprising digitized signals relating to the backscattered millimeter-radar waves;
generate from the received data a detection result including digitized signals of the received data processed to form measured values; and
send the detection result to the microcontroller;
wherein the microcontroller is further configured to:
receive the detection result from the signal processor; and
determine a frequency-dependent reflection coefficient for the specified frequency range using the detection result.

6. The device as claimed in claim 5, wherein:
the microcontroller is further configured to send control data to the signal processor;
the control data comprises an instruction for the irradiation of frequency-modulated millimeter-radar waves in the specified frequency range;
the signal processor is configured to:
receive the control data from the microcontroller;
generate a control signal from the received control data, wherein the control signal comprises at least one variable selected from the group consisting of: a frequency, a frequency deviation, and a modulation method; and
send the control signal to the radar sensor chip;
the radar sensor chip is further configured to:
receive the control signal from the signal processor;
as a result of receiving the control signal, irradiate frequency-modulated millimeter-radar waves in the specified frequency range into the measuring region; and
irradiation occurs as a function of the control signal.

7. The device as claimed in claim 1, wherein:
the radar sensor chip includes a transmitting antenna configured to irradiate frequency-modulated millimeter-radar waves in a specified frequency range into the measuring region.

8. The device as claimed in claim 1, wherein the radar sensor chip comprises a receiving antenna configured to receive millimeter-radar waves backscattered at the mixture.

9. The device as claimed in claim 1, further comprising a radar wave-absorbing layer arranged on an outer wall of the pipe section.

10. The device as claimed in claim 9, wherein the radar wave-absorbing layer comprises a layer of radar wave-absorbing foam.

11. The device as claimed in claim 9, wherein the radar wave-absorbing layer comprises a layer of radar wave-absorbing material including balls coated with carbonyl iron.

12. The device as claimed in claim 9, wherein the radar wave-absorbing layer comprises a layer of radar wave-absorbing polyurethane mixed with balls of carbonyl iron and/or graphite.

13. The device as claimed in claim 1, wherein the radar sensor system is further configured to irradiate frequency-modulated millimeter-radar waves with wavelengths between three and seventeen millimeters in a specified frequency range via the into the measuring region.

14. A method for determining the mixing ratio of a mixture of at least two different fluids, the method comprising:
irradiating continuously frequency-modulated millimeter-radar waves millimeter-radar waves with at least two different frequencies in a measuring region containing the mixture during a measuring process;
receiving continuously frequency-modulated millimeter-radar waves backscattered by the mixture;
determining a frequency-dependent reflection coefficient using the continuously frequency-modulated millimeter-radar waves backscattered at the at least two different frequencies; and
calculating the mixing ratio from the determined reflection coefficient.

15. The method as claimed in claim 14, the method further comprising:
continuous wave irradiating using a transmitting antenna signal with millimeter-radar waves into the measuring region during the measuring process, wherein the irradiated millimeter-radar waves have a specified frequency deviation;
receiving correspondingly frequency-modulated millimeter-radar waves backscattered by the mixture using a receiving antenna signal;
mixing the transmitting antenna signal with the receiving antenna signal to form an intermediate frequency signal;
transforming the intermediate frequency signal into an associated frequency spectrum; and
determining the mixing ratio from the frequency spectrum.
